(19) United States
(12) Patent Application Publication
(10) Pub. No.: US 2026/0094715 A1
(43) Pub. Date: Apr. 2, 2026

(54) MULTI-MODALITY ANOMALY DETECTION USING FUSED MODELS

(71) Applicant: NEC Laboratories America, Inc., Princeton, NJ (US)

(72) Inventors: Junxiang Wang, Plainsboro, NJ (US); Zhengzhang Chen, Princeton Junction, NJ (US); Haifeng Chen, West Windsor, NJ (US); Xu Zheng, Miami, FL (US)

(21) Appl. No.: 19/339,815

(22) Filed: Sep. 25, 2025

Related U.S. Application Data
(60) Provisional application No. 63/701,628, filed on Oct. 1, 2024.

(51) Int. Cl. G16H 50/20; G16H 50/70

(52) U.S. Cl. CPC G16H 50/20; G16H 50/70

(57) ABSTRACT

Systems and methods for multi-modality anomaly detection using artificial intelligence models such as fused models. Metric data and log data obtained from a monitored entity can be encoded into metric representations and log representations by utilizing transformer encoders of a cross-joint variational autoencoder (CJVAE). The metric representations and the log representations can be fused into a joint context representation by utilizing a fusion transformer encoder of the CJVAE. The joint context representation can be decoded by utilizing transformer decoders of the CJVAE to reconstruct the metric representations and the log representations. An anomaly for the monitored entity can be detected by aggregating detection results from the CJVAE based on the metric representations and the log representations, a metric-specific detection result from a metric detector, and a log-specific detection result from a log detector to resolve determined issues of the monitored entity caused by the anomaly.

### RELATED APPLICATION INFORMATION

This application claims priority to U.S. Provisional App. No. 63/701,628, filed on Oct. 1, 2024, incorporated herein by reference in its entirety.

### BACKGROUND

### Technical Field

The present invention relates to anomaly detection using artificial intelligence (AI) models and more particularly to multi-modality anomaly detection using fused models.

### Description of the Related Art

Anomaly detection is an unsupervised learning problem investigated for decades with the goal of finding unusual patterns or behaviors that deviate from expected system performance. It encompasses a wide range of applications, including fraud detection in financial transactions, cyber intrusion detection, and machinery fault diagnosis. The accuracy of anomaly detection is proportional to the accuracy of the system used.

### SUMMARY

According to an aspect of the present invention, a method is provided, including, encoding metric data and log data obtained from a monitored entity into metric representations and log representations by utilizing transformer encoders of a cross-joint variational autoencoder (CJVAE), fusing the metric representations and the log representations into a joint context representation by utilizing a fusion transformer encoder of the CJVAE, decoding the joint context representation by utilizing transformer decoders of the CJVAE to reconstruct the metric representations and the log representations, and detecting an anomaly for the monitored entity by aggregating detection results from the CJVAE based on the metric representations and the log representations, a metric-specific detection result from a metric detector, and a log-specific detection result from a log detector to resolve determined issues of the monitored entity caused by the anomaly.

According to another aspect of the present invention, a system is provided, the system including a memory device, one or more processor devices operatively coupled with the memory device to perform operations including, encoding metric data and log data obtained from a monitored entity into metric representations and log representations by utilizing transformer encoders of a cross-joint variational autoencoder (CJVAE), fusing the metric representations and the log representations into a joint context representation by utilizing a fusion transformer encoder of the CJVAE, decoding the joint context representation by utilizing transformer decoders of the CJVAE to reconstruct the metric representations and the log representations, and detecting an anomaly for the monitored entity by aggregating detection results from the CJVAE based on the metric representations and the log representations, a metric-specific detection result from a metric detector, and a log-specific detection result from a log detector to resolve determined issues of the monitored entity caused by the anomaly.

According to yet another aspect of the present invention, a non-transitory computer program product including a computer-readable storage medium including a program code, wherein the program code when executed on a computer causes the computer to perform, encoding metric data and log data obtained from a monitored entity into metric representations and log representations by utilizing transformer encoders of a cross-joint variational autoencoder (CJVAE), fusing the metric representations and the log representations into a joint context representation by utilizing a fusion transformer encoder of the CJVAE, decoding the joint context representation by utilizing transformer decoders of the CJVAE to reconstruct the metric representations and the log representations, and detecting an anomaly for the monitored entity by aggregating detection results from the CJVAE based on the metric representations and the log representations, a metric-specific detection result from a metric detector, and a log-specific detection result from a log detector to resolve determined issues of the monitored entity caused by the anomaly.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram showing a system for multi-modality anomaly detection using fused models, in accordance with an embodiment of the present invention;

FIG. 2 is a block diagram showing a computer system for multi-modality anomaly detection using fused models, in accordance with an embodiment of the present invention;

FIG. 3 is a block diagram showing a hardware and software components of the system for multi-modality anomaly detection using fused models, in accordance with an embodiment of the present invention;

FIG. 4 is a block diagram showing hardware and software components of the cross joint variational autoencoder, in accordance with an embodiment of the present invention; and

FIG. 5 is a flow diagram showing a method for multi-modality anomaly detection using fused models, in accordance with an embodiment of the present invention.

### DETAILED DESCRIPTION OF PREFERRED EMBODIMENTS

In accordance with embodiments of the present invention, systems and methods are provided for multi-modality anomaly detection using fused models.

In an embodiment, metric data and log data obtained from a monitored entity can be encoded into metric representations and log representations by utilizing transformer encoders of a cross-joint variational autoencoder (CJVAE). The metric representations and the log representations can be fused into a joint context representation by utilizing a fusion transformer encoder of the CJVAE. The joint context representation can be decoded by utilizing transformer decoders of the CJVAE to reconstruct the metric representations and the log representations. An anomaly for the monitored entity can be detected by aggregating detection results from the CJVAE based on the metric representations and the log representations, a metric-specific detection result from a metric detector, and a log-specific detection result from a log detector to resolve determined issues of the monitored entity caused by the anomaly.

Anomaly detection is crucial in IT system operation to prevent issues from escalating and causing severe failures. Effective anomaly detection involves monitoring two primary types of data: metrics and logs.

Metrics are time series consisting of regularly sampled scalar values. They can include measurements such as business KPIs (e.g., transaction success rate), resource utilization (e.g., CPU utilization), and hardware conditions (e.g., GPU temperature).

Logs are sequences of text messages with irregular timestamps, recording various events generated by users, systems, applications, or hardware. These messages can be structured (with fixed templates that can be parsed for key-value pairs) or unstructured (comprising arbitrary natural language sentences).

In practice, metrics and logs are often correlated, and a comprehensive understanding of system status requires analyzing both data types together. However, other anomaly detection methods tend to focus on only one type of data, leading to inaccurate context interpretation and false alerts. For instance, a sudden increase in CPU usage may be normal if a large application was launched but could be abnormal if no such activity is recorded in logs. Conversely, a spike in router events might be benign if accompanied by an increase in network traffic metrics but could indicate a hardware failure if traffic metrics remain normal.

A significant challenge of existing solutions is the difficulty in modeling the interaction between time-series metrics and logs. This challenge arises from inconsistent predictions across these data types. Anomalies may be indicated by either time-series data or log entries, but traditional models often fail to integrate these signals effectively. This inconsistency complicates the creation of a unified model capable of accurately identifying anomalies that span both metrics and logs.

Consider a scenario in a data center where a server experiences a sudden spike in CPU usage (a time-series anomaly) and, simultaneously, an error message is logged indicating a possible hardware failure (a log anomaly). Traditional models might detect the spike in CPU usage as an anomaly but fail to correlate it with the error message, or vice versa. This lack of correlation can lead to missed detections or false positives, as the models do not consider the interaction between the two types of data. Effectively modeling such interactions is crucial for accurate anomaly detection.

To address this challenge, the present embodiments provide an ensemble method for multi-modal anomaly detection. The ensemble method includes three specialized components: a metric detector, a log detector, and a combined metric-log detector.

The metric detector can utilize a forecasting-based model such as a LSTM (Long Short-Term Memory) model to capture temporal patterns and detect anomalies in metric data. The anomaly score is the mean square error between predictions and the true values. If the anomaly score is above the threshold, then it is considered as an anomaly for the metric detector.

The log detector can employ a forecasting based method, such as DeepLog, to analyze log sequences and identify anomalous log entries. The anomaly score can refer to the probabilities that true event types are beyond scope of the pool of the predicted event types. If the anomaly score is above the threshold, then it is considered as an anomaly for the log detector.

The metric-log detector can include a cross joint Variational Autoencoder (CJVAE) model to simultaneously process and detect anomalies across both metric and log data, leveraging the interdependencies between them. The CJVAE is a reconstruction-based method, and the anomaly score can include the normalized sum of errors between reconstructed metric-log pairs and the true metric-log pairs. If the anomaly score is above the threshold, then it is considered as an anomaly for the metric-log detector.

This integrated approach enhances detection accuracy by effectively modeling the interactions between time-series and log data, providing a comprehensive solution for anomaly detection in multi-modal data environments. The present embodiments improve model robustness and flexibility by isolating different anomaly types through specialized detectors. Additionally, the modular design allows for parallel processing of metric and log modalities, which can optimize computational processing efficiency. The use of compact latent representations in the variational autoencoder (VAE) also reduces storage and computational overhead during inference.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

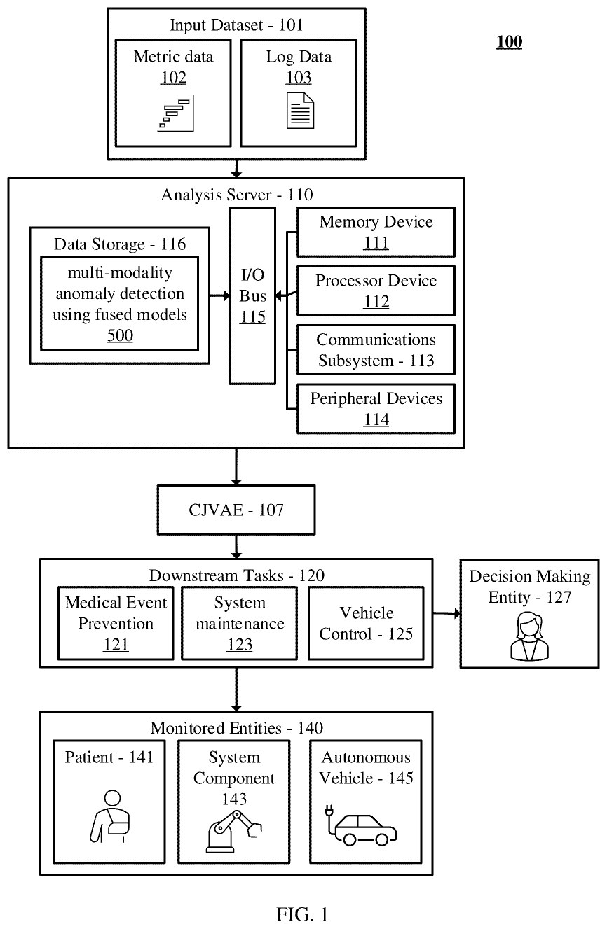

Referring now in detail to the figures in which like numerals represent the same or similar elements and initially to FIG. 1, a block diagram showing a system for multi-modality anomaly detection using fused models, in accordance with an embodiment of the present invention.

In an embodiment, system 100 can utilize an analysis server 110 that implements multi-modality anomaly detection using fused models 400 to process input dataset 101 and train a cross-joint variational autoencoder (CJVAE) 107 to perform downstream tasks 120 to assist the decision-making process of a decision-making entity 127 for monitored entities 140.

The input dataset 101 can include metric data 102 and log data 103 that can be obtained from monitored entities 140. The monitored entities 140 can include a patient 141, system component 143, and an autonomous vehicle 145.

Downstream tasks 120 can include medical event prevention 121, system maintenance 123, and vehicle control 125.

In medical event prevention 121, an input dataset 101 (e.g., x-ray images, vital sign readings, body scans, etc.) of a patient 141 can be processed by the CJVAE 107 to detect anomalies (e.g., abnormal increase in vital signs, abnormal increase in cell count, abnormal increase in blood sugar levels, etc.) from the input dataset 101 and prevent an undesirable medical event (e.g., hypertension, cancer, diabetes, etc.). The decision-making entity 127 (e.g., healthcare professional) responsible for the patient 141 can be notified of the detected anomalies of the CJVAE 107 to help the decision-making process of the decision-making entity 127 such as updating a medical diagnosis for the patient 141, recommending lifestyle choices for the patient 141, administering a medical treatment for a detected disease (e.g., insulin for diabetes, etc.).

In system maintenance 123, input dataset 101 (e.g., system logs, test cases, hardware status images, etc.) related to the system component 143 (e.g., request server of a distributed computing application) can be processed by the CJVAE 107 to detect system anomalies (e.g., abnormal increase in requests for a server, abnormal dip in storage resources, abnormal increase in processing power consumption, etc.). With the CJVAE 107, autonomous system maintenance can be performed on the system component 143 such as adding bandwidth, blocking packets from an identified internet protocol (IP) address to resolve malicious attacks, restarting hardware, etc. In another embodiment, the decision-making entity 127 (e.g., information technology (IT) professional) responsible for the system component 143 can be notified of the detected anomalies of the CJVAE 107 to help the decision-making process of the decision-making entity 127 and verify the autonomous system maintenance performed on the system component 143 through automated decision making.

In vehicle control 125, input dataset 101 (e.g., vehicle part status, traffic scene image, etc.) related to the autonomous vehicle 145 can be processed by the CJVAE 107 to detect system anomalies (e.g., abnormal rise of temperature, abnormal increase in speed, abnormal idling, etc.). A corrective action 130 can be generated by the analytic server 103 which can include the answer to the user queries 128 to control the proper performance of the autonomous vehicle 145. With the CJVAE 107, the autonomous vehicle 145 can be autonomously controlled (e.g., stopping, speeding up, changing direction, etc.) using appropriate control devices (e.g., advanced driver assistance systems, braking device, accelerator device, cooling device, etc.) within the autonomous vehicle. In another embodiment, the decision-making entity 127 (e.g., driver, handler, etc.) responsible for the autonomous vehicle 145 can be notified of the detected anomalies of the CJVAE 107 to help the decision-making process of the decision-making entity 127 and verify the autonomous vehicle control performed on the autonomous vehicle 145 through automated decision making.

The analysis server 110 can include a memory device 111, a processor device 112, a communications subsystem 113, peripheral devices 114, input/output (I/O) bus 115, and data storage 116, that can store program instructions for multi-modality anomaly detection using fused models 400.

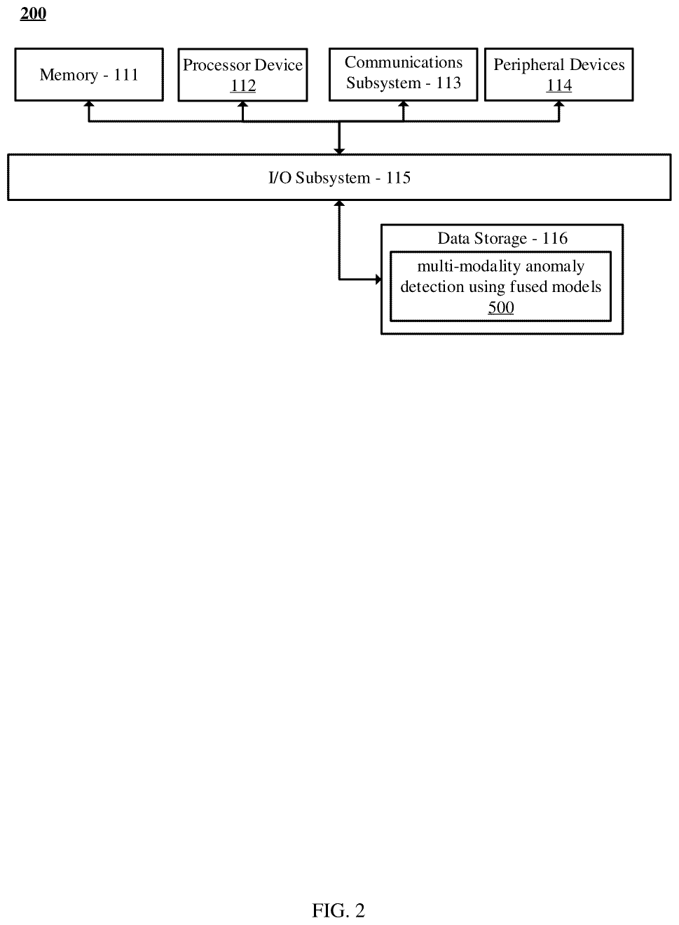

Referring now to FIG. 2, a block diagram showing a computer system for multi-modality anomaly detection using fused models, in accordance with an embodiment of the present invention.

In an embodiment, computing device 200 can be implemented as analysis server 110. The computing device 200 illustratively includes the processor device 112, an input/output (I/O) subsystem 115, a memory 111, a data storage device 116, and a communications subsystem 113, and/or other components and devices commonly found in a server or similar computing device. The computing device 200 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 111, or portions thereof, may be incorporated in the processor device 112 in some embodiments.

The processor device 112 may be embodied as any type of processor capable of performing the functions described herein. The processor device 112 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 111 may be embodied as any type of volatile or non-volatile memory or data storage capable of performing the functions described herein. In operation, the memory 111 may store various data and software employed during operation of the computing device 200, such as operating systems, applications, programs, libraries, and drivers. The memory 111 is communicatively coupled to the processor device 112 via the I/O subsystem 115, which may be embodied as circuitry and/or components to facilitate input/output operations with the processor device 112, the memory 111, and other components of the computing device 200. For example, the I/O subsystem 115 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 115 may form a portion of a system-on-a-chip (SOC) and be incorporated, along with the processor device 112, the memory 111, and other components of the computing device 200, on a single integrated circuit chip.

The data storage device 116 may be embodied as any type of device or devices configured for short-term or long-term storage of data such as, for example, memory devices and circuits, memory cards, hard disk drives, solid state drives, or other data storage devices. The data storage device 116 can store program code for multi-modality anomaly detection using fused models 400. Any or all of these program code blocks may be included in a given computing system.

The communications subsystem 113 of the computing device 200 may be embodied as any network interface controller or other communication circuit, device, or collection thereof, capable of enabling communications between the computing device 200 and other remote devices over a network. The communications subsystem 113 may be configured to employ any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 200 may also include one or more peripheral devices 114. The peripheral devices 114 may include any number of additional input/output devices, interface devices, and/or other peripheral devices. For example, in some embodiments, the peripheral devices 114 may include a display, touch screen, graphics circuitry, keyboard, mouse, speaker system, microphone, network interface, and/or other input/output devices, interface devices, GPS, camera, and/or other peripheral devices.

Of course, the computing device 200 may also include other elements (not shown), as readily contemplated by one of skill in the art, as well as omit certain elements. For example, various other sensors, input devices, and/or output devices can be included in computing device 200, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing device 200 are readily contemplated by one of ordinary skill in the art given the teachings of the present invention provided herein.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

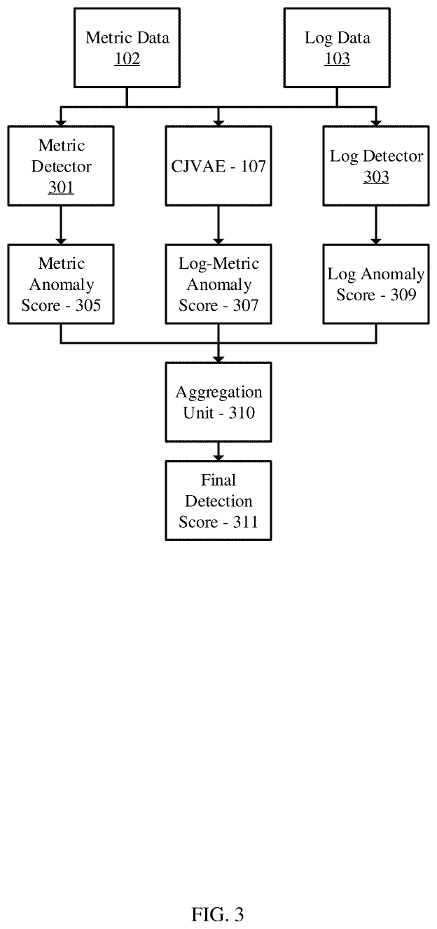

Referring now to FIG. 3, a block diagram showing a hardware and software components of the system for multi-modality anomaly detection using fused models, in accordance with an embodiment of the present invention.

In an embodiment, system 300 can process metric data 102 with a metric detector 301, log data 103 with log detector 303, and both data with CJVAE 107 to detect an anomaly. The metric detector 301 can output a metric anomaly score 305. The log detector 303 can output a log anomaly score 309. The CJVAE 107 can output a log-metric anomaly score 307. An aggregation unit 310 aggregates the metric anomaly score 305, the log anomaly score 309 and the log-metric anomaly score 307 to compute a final detection score 311. If the final detection score 311 is below an anomaly threshold, then an anomaly has been detected.

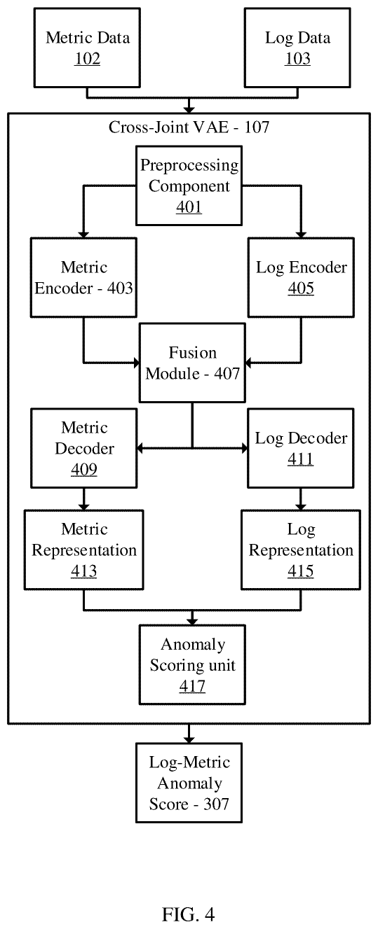

Referring now to FIG. 4, a block diagram showing hardware and software components of the cross joint variational autoencoder, in accordance with an embodiment of the present invention.

In an embodiment, CJVAE 107 can process metric data 102 and log data 103 to compute a log-metric anomaly score 307 that can be aggregated with metric anomaly score 305 and log anomaly score 309 to compute a final detection score 311 and detect anomalies.

CJVAE 107 includes a preprocessing component 401 that preprocesses metric data 102 and log data 103. The preprocessing component 401 can normalize metric data 102 to ensure a consistent data range. The preprocessing component 401 can impute missing values in the metric data 102 with default values. The preprocessing component 401 can train a parser with the log data 103 to construct a parse tree that can map test logs to a template. The preprocessing component 401 can tokenize log data 103 to be mapped using the parse tree. The preprocessing component 401 can resample the metric data 102 and the log data 103 to align the two modalities.

After preprocessing the metric data 102 and the log data 103, a metric encoder 403 can process the metric data 102 and a log encoder 405 can process the log data to generate embeddings. The embeddings can be fused by a fusion module 407 where log data 103 is crossed with metric embeddings and metric data 102 is crossed with log embeddings. The crossed embeddings are processed by the metric encoder 409 and the log decoder 411 to generate metric representation 413 and log representation 415 respectively. The metric encoder 403, the log encoder 405, and the fusion module 407 can utilize neural networks such as transformers.

A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types and may include multiple distinct values. The network can have one input neurons for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

The neural network, such as a multilayer perceptron, can have an input layer of source neurons, one or more computation layer(s) having one or more computation neurons, and an output layer, where there is a single output neuron for each possible category into which the input example could be classified. An input layer can have a number of source neurons equal to the number of data values in the input data. The computation neurons in the computation layer(s) can also be referred to as hidden layers, because they are between the source neurons and output neuron(s) and are not directly observed. Each neuron in a computation layer generates a linear combination of weighted values from the values output from the neurons in a previous layer, and applies a non-linear activation function that is differentiable over the range of the linear combination. The weights applied to the value from each previous neuron can be denoted, for example, by w₁, w₂, . . . wₙ₋₁, wₙ. The output layer provides the overall response of the network to the inputted data. A deep neural network can be fully connected, where each neuron in a computational layer is connected to all other neurons in the previous layer, or may have other configurations of connections between layers. If links between neurons are missing, the network is referred to as partially connected.

Training a deep neural network can involve two phases, a forward phase where the weights of each neuron are fixed and the input propagates through the network, and a backwards phase where an error value is propagated backwards through the network and weight values are updated. The computation neurons in the one or more computation (hidden) layer(s) perform a nonlinear transformation on the input data that generates a feature space. The classes or categories may be more easily separated in the feature space than in the original data space.

An anomaly scoring unit 417 can process the metric representation 413 and the log representation 415 to compute the log-metric anomaly score 307 based on reconstruction of the crossed representations to the metric data 102 and log data 103.

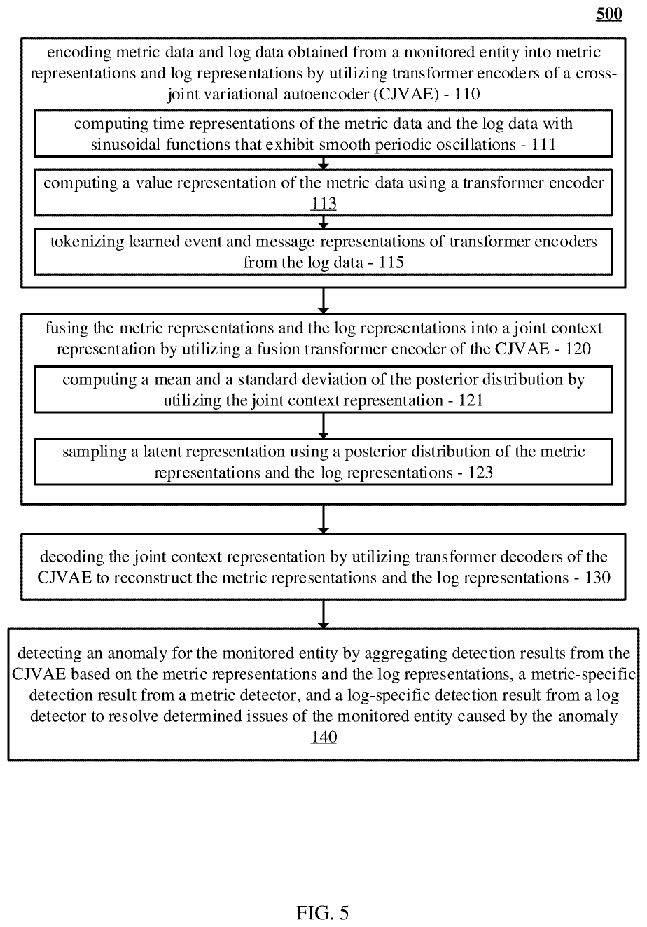

Referring now to FIG. 5, a flow diagram showing a method for multi-modality anomaly detection using fused models, in accordance with an embodiment of the present invention.

In an embodiment, metric data and log data obtained from a monitored entity can be encoded into metric representations and log representations by utilizing transformer encoders of a cross-joint variational autoencoder (CJVAE). The metric representations and the log representations can be fused into a joint context representation by utilizing a fusion transformer encoder of the CJVAE. The joint context representation can be decoded by utilizing transformer decoders of the CJVAE to reconstruct the metric representations and the log representations. An anomaly for the monitored entity can be detected by aggregating detection results from the CJVAE based on the metric representations and the log representations, a metric-specific detection result from a metric detector, and a log-specific detection result from a log detector to resolve determined issues of the monitored entity caused by the anomaly.

In block 510, metric data and log data obtained from a monitored entity can be encoded into metric representations and log representations by utilizing transformer encoders of a cross-joint variational autoencoder (CJVAE);

Metric representation (\tilde{x}ᵢ) of metric data xᵢ and log representations (\tilde{m}ⱼ) of log data mj can be obtained: \tilde{x}ᵢ=g1(t̅₁+x̅ᵢ), \tilde{m}ⱼ=g2(τ̅ⱼ+ūⱼ+m̅ⱼ), where t̅ᵢ and τ̅ⱼ represent the metric time and value encodings, respectively. Similarly, τ̅ⱼ, ūⱼ, and m̅ⱼ represent the time, event, and message encodings of log data, respectively. Both g1 and g2 can utilize transformers.

In another embodiment, additional metadata can be encoded. Examples include machine identifiers (e.g., hostname, server ID), network attributes (e.g., IP address, port number), user context (e.g., user ID, session ID), geolocation data (e.g., region, GPS coordinates), system configuration details (e.g., software version, hardware type), and application-level metadata (e.g., container ID, process name). These attributes can be tokenized and embedded similarly to other inputs, enabling the model to incorporate richer context and detect environment-specific or user-specific anomalies more effectively.

In block 511, time representations of the metric data 102 and the log data 103 can be computed using sinusoidal functions that exhibit smooth periodic oscillations. The metric timestamp tᵢ can be defined as

$${c}_{i , k}^{t} = cos ( \frac{2 π {t}_{i}}{{2}^{i}} ) , {s}_{i , k}^{t} = sin ( \frac{2 π {t}_{i}}{{2}^{i}} ) .$$

The log timestamp tᵢ can be defined as

$${c}_{j , k}^{τ} = cos ( \frac{2 {πτ}_{i}}{{2}^{j}} ) , {s}_{j , k}^{τ} = sin ( \frac{2 {πτ}_{i}}{{2}^{j}} ) .$$

Then time representations can be calculated as follows:

$${\overline{t}}_{i} = {f}_{t} ( {t}_{i} ) = {{W}_{t} [ {c}_{i , 1}^{t} , {s}_{i , 1}^{t} , {c}_{i , 2}^{t} , {s}_{i , 2}^{t} , … , {c}_{i , K}^{t} , {s}_{i , K}^{t} ]}^{T} , \qquad ( 1 )$$

$${\overline{t}}_{j} = {f}_{τ} ( {t}_{j} ) = {{W}_{τ} [ {c}_{i , 1}^{τ} , {s}_{i , 1}^{τ} , {c}_{i , 2}^{τ} , {s}_{i , 2}^{τ} , … , {c}_{i , K}^{τ} , {s}_{i , K}^{τ} ]}^{T} , \qquad ( 2 )$$

(2) where t̅ᵢ and τ̅ⱼ are the time representations of tᵢ and τⱼ, respectively. Wₜ and W_{τ} are learned projection matrices for the metric and log timestamps, and K is the number of sinusoidal function pairs.

In block 513, a value representation x̅ᵢ of metric data xᵢ can be computed:
x̅ᵢ=g3(xᵢ), (3) where g3 is a transformer encoder.

In block 515, learned event and message representations can be tokenized and embedded using the token embedding function e(⋅). These embeddings can be learned from scratch or initialized using a pretrained tokenizer:

$${\overline{u}}_{j} = e ( {u}_{j} ) , \qquad ( 4 )$$

m̅ⱼ=g₄(e(mⱼ)), (5) where ūⱼ and m̅ⱼ are the event and message representations of uⱼ and mⱼ, respectively, and g4 is a transformer encoder.

In block 520, the metric representations and the log representations can be fused into a joint context representation by utilizing a fusion transformer encoder of the CJVAE;

The fusion module 407 can integrate the metric representation \tilde{x}ᵢ and the log representation \tilde{m}ⱼ into a joint context representation: h=g₅({\tilde{x}ᵢ}ᵢ₌₁°{\tilde{m}ⱼ}ⱼ₌₁), where g₅ is a fusion transformer encoder, ⋅ is the concatenation along the time dimension, and h is a contextual representation.

In block 521, a joint context representation h can be used to compute the mean u and the standard deviation o of a posterior distribution q(z|X, M). In another embodiment, other statistical measures and distributional assumptions can enhance flexibility and robustness. For instance, higher-order moments such as skewness and kurtosis can help model asymmetry and tail behavior in latent representations. Alternatively, one can use non-Gaussian priors, such as Student's t-distributions (for heavier tails) or mixture models (e.g., Gaussian Mixture Models), which better capture multi-modal or outlier-prone data. In more advanced setups, normalizing flows or variational inference with implicit distributions can be used to learn complex posterior shapes. Moreover, quantile-based thresholds or empirical cumulative distribution functions (ECDFs) can replace fixed assumptions entirely, allowing anomaly boundaries to be data-driven. These enhancements improve the expressiveness of the posterior and can lead to more accurate and calibrated anomaly scores.

In block 523, the posterior distribution can be utilized to sample a latent representation z for metric and log reconstructions: [μ; σ]=Wh+b, q(z|X, M)=N (z; μ, σI) where W and b are the weight and bias of the linear layer.

In block 530, the joint context representation can be decoded by utilizing transformer decoders of the CJVAE to reconstruct the metric representations and the log representations.

Given a sampled latent z˜q(z|X, M), the metric decoder 409 and the log decoder 411 can reconstruct the metric and the log from z, which can be achieved with transformer G₁ and G₂. Specifically, the reconstructed metric can be computed with: \hat{x}ᵢ=G₁(z,{uⱼ}ⱼ₌₁), where z and uⱼ are aligned by the cross-attention in G₁ to match the metric representation in z. Similarly, the reconstructed log is û′ⱼ=G₂ (z,{xᵢ}ᵢ₌₁)∈[0, 1], ûⱼ=argmax û′ⱼ, where û′ⱼ is the probability distribution of the reconstructed event type, and ûⱼ is the reconstructed event type.

In block 540, an anomaly for the monitored entity can be detected by aggregating detection results from the CJVAE based on the metric representations and the log representations, a metric-specific detection result from a metric detector, and a log-specific detection result from a log detector to resolve determined issues of the monitored entity caused by the anomaly.

Let

$$\hat{X} = {\{ {t}_{i} , {\hat{x}}_{i} \}}_{i = 1}^{T} and \hat{M} = {\{ {τ}_{j} , {\hat{u}}_{j} , {m}_{j} \}}_{j = 1}^{N}$$

as the reconstructed metric and log sequences, respectively. Then the objective can be formulated mathematically as follows: [symbol] = [symbol] ₘₑₜ(X, \hat{X})+α [symbol] _{log}(M, \hat{M})+β [symbol] _{reg}(X, M). where [symbol] ₘₑₜ(X, \hat{X}) is the reconstruction loss of the metric, which is achieved by the Mean Squared Error (MSE), [symbol] _{log} (M, M) is the reconstruction loss of the log, which is achieved by the Cross-Entropy loss, [symbol] _{reg} (X, M) is the regularization loss, which is achieved by the Kullback-Leibler (KL) divergence, α>0 and β>0 are two hyperparameters to balance three terms.

The anomaly score is defined as the sum of two reconstruction losses. The threshold is defined as the mean plus three standard deviations of anomaly scores of all training samples.

Aside from the metric-log detector, two individual metric and log detectors are utilized to detect anomalies based on the single modality, providing further support for the metric-log detector. The thresholds of the two detectors are defined as the 95th percentile anomaly scores of all training samples. Specifically, the metric detector can be set to the Peak Over Threshold (POT) model, which is a statistical approach based on extreme value theory. The POT model can learn the behavior of extreme events by fitting them into the generalized Pareto distribution, on which the anomaly score is based.

For the log detector, a combination of a frequency-based detector and a Principal Component Analysis (PCA) model can be employed. The frequency-based log detector can check whether log time-series exhibit periodic patterns. The PCA model transforms a sequence of event types into continuous feature representations (e.g., occurrence counts or Term Frequency-Inverse Document Frequencies (TF-IDFs) of event types). The anomaly score is the reconstruction error between the reconstructed representations and the actual representations. The anomaly score of the log detector is the average of the normalized anomaly scores from the frequency-based log detector and the PCA detector (i.e., here the normalized anomaly score is the original anomaly score divided by the threshold).

Outputs from three independent detectors are aggregated to compute the final detection score, and a simple majority voting strategy can be applied. A sample is flagged as an anomaly if two out of three detectors label it as an anomaly. In another embodiment, alternative aggregation methods can be employed to enhance anomaly decision-making. Weighted averaging assigns confidence scores or reliability weights to each detector based on validation performance or historical accuracy. Bayesian model averaging combines the outputs probabilistically, incorporating prior knowledge and uncertainty estimates. Stacking (meta-learning) uses a separate model (e.g., logistic regression or a neural network) trained on the outputs of individual detectors to learn optimal combination strategies. Other methods include max-pooling or min-pooling, which are useful in high-sensitivity or high-specificity settings, and rule-based logic, which allows domain experts to define conditional aggregation rules. These methods offer more nuanced integration of diverse detector outputs and can be tuned to specific application requirements such as reducing false positives or prioritizing rare but critical anomalies.

The present embodiments enables effective and robust anomaly detection not only because of the ensemble technique, but also due to the diversity of our detectors: our metric-log detector is a nonlinear reconstruction-based model, our metric detector is a probabilistic approach, and our log detector is based on frequency and low-dimensionality. Due to this diversity, the present embodiments can effectively capture different types of anomalies (e.g., abnormal frequencies of event types, unexpected log sequence patterns, and extreme metric values).

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A method, comprising:
encoding metric data and log data obtained from a monitored entity into metric representations and log representations by utilizing transformer encoders of a cross-joint variational autoencoder (CJVAE);
fusing the metric representations and the log representations into a joint context representation by utilizing a fusion transformer encoder of the CJVAE;
decoding the joint context representation by utilizing transformer decoders of the CJVAE to reconstruct the metric representations and the log representations; and
detecting an anomaly for the monitored entity by aggregating detection results from the CJVAE based on the metric representations and the log representations, a metric-specific detection result from a metric detector, and a log-specific detection result from a log detector to resolve determined issues of the monitored entity caused by the anomaly.

2. The method of claim 1, wherein encoding the metric data and the log data further comprises computing time representations of the metric data and the log data with sinusoidal functions that exhibit smooth periodic oscillations.

3. The method of claim 1, wherein encoding the metric data and the log data further comprises computing a value representation of the metric data using a transformer encoder.

4. The method of claim 1, wherein encoding the metric data and the log data further comprises tokenizing learned event and message representations of transformer encoders from the log data.

5. The method of claim 1, wherein fusing the metric representations and the log representations further comprises sampling a latent representation using a posterior distribution of the metric representations and the log representations.

6. The method of claim 5, wherein fusing the metric representations and the log representations further comprises computing a mean and a standard deviation of the posterior distribution by utilizing the joint context representation.

7. The method of claim 1, further comprising notifying a decision-making entity about the anomaly detected from metric and log data obtained from patient data through automated decision making.

8. A system, comprising:
a memory device;
one or more processor devices operatively coupled with the memory device to perform operations including:
encoding metric data and log data obtained from a monitored entity into metric representations and log representations by utilizing transformer encoders of a cross-joint variational autoencoder (CJVAE);
fusing the metric representations and the log representations into a joint context representation by utilizing a fusion transformer encoder of the CJVAE;
decoding the joint context representation by utilizing transformer decoders of the CJVAE to reconstruct the metric representations and the log representations; and
detecting an anomaly for the monitored entity by aggregating detection results from the CJVAE based on the metric representations and the log representations, a metric-specific detection result from a metric detector, and a log-specific detection result from a log detector to resolve determined issues of the monitored entity caused by the anomaly.

9. The system of claim 8, wherein encoding the metric data and the log data further comprises computing time representations of the metric data and the log data with sinusoidal functions that exhibit smooth periodic oscillations.

10. The system of claim 8, wherein encoding the metric data and the log data further comprises computing a value representation of the metric data using a transformer encoder.

11. The system of claim 8, wherein encoding the metric data and the log data further comprises tokenizing learned event and message representations of transformer encoders from the log data.

12. The system of claim 8, wherein fusing the metric representations and the log representations further comprises sampling a latent representation using a posterior distribution of the metric representations and the log representations.

13. The system of claim 12, wherein fusing the metric representations and the log representations further comprises computing a mean and a standard deviation of the posterior distribution by utilizing the joint context representation.

14. The system of claim 8, further comprising notifying a decision-making entity about the anomaly detected from metric and log data obtained from patient data through automated decision making.

15. A non-transitory computer program product comprising a computer-readable storage medium including a program code, wherein the program code when executed on a computer causes the computer to perform:
encoding metric data and log data obtained from a monitored entity into metric representations and log representations by utilizing transformer encoders of a cross-joint variational autoencoder (CJVAE);
fusing the metric representations and the log representations into a joint context representation by utilizing a fusion transformer encoder of the CJVAE;
decoding the joint context representation by utilizing transformer decoders of the CJVAE to reconstruct the metric representations and the log representations; and
detecting an anomaly for the monitored entity by aggregating detection results from the CJVAE based on the metric representations and the log representations, a metric-specific detection result from a metric detector, and a log-specific detection result from a log detector to resolve determined issues of the monitored entity caused by the anomaly.

16. The non-transitory computer program product of claim 15, wherein encoding the metric data and the log data further comprises computing time representations of the metric data and the log data with sinusoidal functions that exhibit smooth periodic oscillations.

17. The non-transitory computer program product of claim 15, wherein encoding the metric data and the log data further comprises computing a value representation of the metric data using a transformer encoder.

18. The non-transitory computer program product of claim 15, wherein encoding the metric data and the log data further comprises tokenizing learned event and message representations of transformer encoders from the log data.

19. The non-transitory computer program product of claim 15, wherein fusing the metric representations and the log representations further comprises sampling a latent representation using a posterior distribution of the metric representations and the log representations.

20. The non-transitory computer program product of claim 15, further comprising notifying a decision-making entity about the anomaly detected from metric and log data obtained from patient data through automated decision making.
